(19) United States
(12) Patent Application Publication
(10) Pub. No.: US 2003/0232431 A1
(43) Pub. Date: Dec. 18, 2003

(54) Cellular transplantation for heart regeneration

(76) Inventor: Peter K. Law, Germantown, TN (US)

Correspondence Address: HELLER EHRMAN WHITE & MCAULIFFE LLP, 1666 K STREET,NW, SUITE 300, WASHINGTON, DC, 20006, US

(21) Appl. No.: 10/403,520

(22) Filed: Apr. 1, 2003

Related U.S. Application Data
(60) Provisional application No. 60/368,563, filed on Apr. 1, 2002.

(51) Int. Cl. C12N 5/08

(52) U.S. Cl. 435/366

(57) ABSTRACT

Myoblast cells obtained by culturing, particularly from satellite cells or other progenitor cells, are transplanted into tissue such as diseased heart tissue to form healthy repair tissue and reverse disease. This technique can be carried out in various ways and preferably includes a cellular integration factor to assist cellular survival, integration and longevity into the treated organ. Angiogenesis factors such as vascular endothelial growth factor are particularly preferred and may be transgenically expressed by the transplanted cell. Other factors that may be used to augment the procedure include migratory and scaffolding molecules. The methods and materials are particularly useful in combination with an automated cell processor and an automated catheter delivery system. The materials and methods for their use may be applied to the prophylaxis and therapy of damaged hearts, using cells originally obtained from the patient, another human, or another animal.

### FIELD OF THE INVENTION

The Invention relates to cell therapy of heart and particularly to the use of myogenic skeletal tissue derived cells for prophylactic and therapeutic treatment, and chemical agents that facilitate such treatment.

### BACKGROUND

Heart muscle degeneration is a leading cause of debilitation and death in humans. A common pathway underlies congenital and infectious cardiomyopathies, myocardial infarction, congestive heart failure, angina, coronary artery disease and peripheral vascular disease, all of which constitute the cardiovascular diseases. Global healthcare spending heart muscle degeneration topped $280 billion in 2001. In the United States alone, approximately $186 billion is spent every year in treating some 60 million cardiovascular disease patients. However, about 50% of the patients suffering congestive heart failure die within 5 years of diagnosis.

Heart muscle degeneration cascades with cardiomyocyte membrane leakage, uncontrolled Ca²⁺ influx, mitochondrial ATP shutdown, inability to exude Ca²⁺ through the cell surface and to reabsorb Ca²⁺ into the sarcoplasmic reticulum, myofibrillar hypercontracture and disarrangement. Apoptosis ensues and fibroblasts proliferate and infiltrate. The heart muscle, which is populated by live cardiomyocytes with proteinaceous contractile filaments such as myosin, actin, troponin, tropomyosin, is partially occupied by fibrous scars that are incapable of electric conduction, mechanical contraction and revascularization. These scars continue to exert a negative compliance on the heart and the circulation despite remodeling occurs after a myocardial infarction.

This degeneration results in the loss of live cardiomyocytes, contractile filaments, contractility, heart function and healthy circulation. The damaged heart responds by cell division of cardiomyocytes. However such regenerative capacity is hardly significant. Cardiomyocytes in culture will undergo no more than three to five divisions, generally yielding an insufficient number of cells to repopulate a myocardial infarct. Cardiomyocytes do not multiply significantly because human telomeric DNA repeats (Ishikawa, et al., Molecular Cell Biology, 13, 4301-4310, 1993) in terminally differentiated cells are minimal. As a result, without significant mitotic activity, surviving cardiomyocytes cannot provide enough new cells to deposit the contractile filaments necessary to maintain normal heart function.

One cellular strategy suggested to combat this problem has been to transplant stem cells into the heart. Stem cell technology has gained much attention due to the controversy of utilizing cells from human embryos. More critically, scientists generally do not know the specific factor(s) that trigger stem cells to differentiate only into heart muscle cells, and not into other cell types. Until such knowledge becomes available, stem cell transplants into the heart may result in bony, cartilageous, fatty and fibrotic elements that are detrimental to heart function. Being pluripotent, embryonic or adult stem cells exhibit uncontrolled differentiation into various lineages to produce bone, cartilage, fat, connective tissue, skeletal and heart muscles. Until scientists can accurately define the specific transcriptional factors and pathway to guide stem cell differentiation into cardiomyocytes, the use of stem cell injection into the human heart would have a higher risk-benefit ratio than from using myoblasts. Accordingly, these procedures have many obstacles to their widespread use.

Another suggested strategy has been the use of transmyocardial revascularization. However, heart muscle cells are terminally differentiated and do not divide significantly to regenerate damaged heart muscle. And transplants of foreign heart parts generally requires the use of lifelong immunosuppressants, which pose major infection risks and subsequent death of heart transplant patients. In this context, the degenerative heart also transmits biochemical signals to recruit stem cells, from the stroma and the bone marrow, in an attempt to repair the muscle damage on its own. However, much of the recruited stem cells differentiate to become fibroblasts instead of cardiomyocytes, thus forming fibrous scars and not contractile filaments.

A major problem with cellular therapies, accordingly, has been the inability to add new cells of the right amount and type to damaged heart tissue. Therefore, despite the claimed success of transmyocardial revascularization using laser, angiogenic factors and genes, the damaged myocardium needs additional live cells to deposit contractile filaments to regain heart function, preferably before fibroblast infiltration which leads to scar formation.

Other therapies to address the damaged heart problem include the use of drugs such as angiotensin converting enzyme (ACE) inhibitors and beta-blockers, which generally treat symptoms and provide temporary relief. Further acute measures that save lives include implantation of a pacemaker, cardioverter defibrillator (ICD) and left ventricular assisted devices (LVAD). More recently, injections of angiogenic factor(s) or VEGF genes have found to produce an increase in the number of capillaries. However, none of these treatments can add contractile filaments that are necessary to regain heart contractility lost in heart patients.

### SUMMARY OF THE INVENTION

The above summarized problems were alleviated by a series of techniques and materials involving the culture of tissue biopsy specimens to form cell cultures for transplant, transplanting cultured cells into a heart, and the use of other factors such as angiogenesis factors in combination thereto. The added factors may be used, for example, before, during and/or after transplantation to prepare new heart vessels for the transplanted cells, target the transplanted cells more specifically, and to help bind the transplanted cells. In one embodiment, the factor(s) are made by the transplanted cells themselves. In another embodiment one or more factors are supplied to the heart directly, and optionally are complexed to other material in a slow release form.

Embodiments provide improved integration and survival of transplanted cells. In one embodiment, techniques and materials provided herein allow the use of smaller numbers of transplanted cells, for improved efficacy and lower cost. In another embodiment, techniques and materials provide greater regenerative capacity, for healthy hearts, healthy muscles, as well as for diseased hearts and diseased muscles. In yet another embodiment improved integration of myoblasts is achieved. Other advantages will be appreciated by a reading of the specification.

An embodiment of the invention is a method for producing cardiomyocytes capable of proliferation, comprising providing cardiomyocyte cells; providing myoblast cells; and mixing the cells under in vitro or in vivo conditions that allow cell fusion of cardiomyocyte cells with myoblast cells to form heterokaryotic cardiomyocytes. Another embodiment is a method of replenishing degenerated and degenerating cardiomyocytes of a patient with heart disease, comprising providing heterokaryotic cardiomyocytes capable of developing desmosomes and gap junctions; and administering the heterokaryotic cardiomyocytes through a catheter pathway.

Yet another embodiment is a composition of cells useful for repair of damaged heart muscle, comprising myoblasts that have been transgenically transformed to express a cellular integration factor selected from the group consisting of an angiogenesis factor, vascular endothelial growth factor, fibroblast growth factor, platelet derived growth factor, angiogenin, pleiotrophin, and interleukin-8. Yet another embodiment is a composition of cells useful for repair of damaged heart muscle, comprising myoblasts and an effective amount of a cellular integration factor selected from the group consisting of an angiogenesis factor, vascular endothelial growth factor, fibroblast growth factor, platelet derived growth factor, angiogenin, pleiotrophin, and interleukin-8a migration factor, a scaffolding protein, PDGF, HGF, fibronectin, MMP-1, MMP-2, laminin, laminin-1, fibronectin, type I collagen, type II collagen, type IV collagen, thrombospondin-1, lecithin-oxytetracycline-collagen matrix, a galactin, galectin-1, vitronectin, and von Willebrand protein. Further embodiments will be appreciated by a reading of the specification.

### DETAILED DESCRIPTION

Insights were obtained through extensive work in the field of myoblast transplantation therapy that allow the extension of this basic technique to the prophylactic and therapeutic fortification of heart muscle with myoblasts obtained from skeletal muscle. For example, it is realized that newly formed myotubes have to be vascularized and innervated within 10 days, or they will perish. Successfully innervated and vascularized, the transplanted cells deposit actin, myosin, troponin and tropomyosin that eventually organize into sarcomeres, the structural units of muscle contraction. This maturation process takes approximately three months. Unfortunately, previous studies have indicated that a large portion of transplanted cells fail to integrate successfully. In contrast, embodiments that employ one or more techniques and materials described here, provide more advantageous nutrient (including oxygen) supplementation of transplanted cells, greater targeting, and greater survivability of transplanted cells.

Embodiments of the invention span a range of materials and methods. In desirable embodiments, allographs are made by initial cell sampling of skeletal tissue from another human donor to prepare cells for transplant into another. An advantage of this technique is that a more reproducible cell culture technique may be used for a more standardized procedure that may require fewer calibration and control tests for treating multiple patients. Furthermore, this procedure allows large scale up at central cell manufacturing locations that may utilize, for example an automated cell processor for lowered costs and greater availability of the technique to patients. Another advantage is that genetically desirable cells may be used for implantation into genetically weak recipients. Yet another advantage is that transgenic manipulation of a single sample can be used to treat multiple patients, for improved quality control and reduced costs. In yet another embodiment, specific modification of tissue rejection antigens on a chosen cell sample may be carried out to remove or alleviate transplantation antigens for a standard cell type.

Another embodiment of the invention uses autograft transplantation. In this technique, a biopsy such as a muscle sample is taken from a patient, cells are grown up from the satellite cells (or other muscle progenitor cells that may be present) and then re-implanted into the desirable area of the heart, other muscle or other tissue. This technique is particularly desirable where the patient is young, has genetically normal skeletal tissue for sampling, and ample time exists to establish a large culture from the sampled cells. This technique is particularly desirable where it is desired to avoid possible complications of tissue rejection.

In both allograph and autograft methods, the technology of myoblast transfer therapy (MTT) desirably is used as described by the publications of Dr. Peter Law, who has treated degenerative, genetic diseases such as muscle scerosis with success. See P K Law, et al., “Myoblast Transfer as a Platform Technology of Gene Therapy”, Gene Therapy & Molecular Biology, 1 (1998), pp. 345-363. MTT is a platform technology of cell transplantation, nuclear transfer and tissue engineering. See P K Law, “Myoblast Transfer as a Platform Technology of Gene Therapy”, Regulatory Affairs Focus, (Technology), 4 (1999), pp. 25-27 and P K Law, “Nuclear Transfer and Human Genome Therapy”, Business Briefing—Future Drug Discovery (Genomics), (December, 2001), pp. 38-42. Without wishing to be bound by any one theory for this embodiment of the invention, it is thought that satellite cells exist between the basement membrane and the plasma membrane of skeletal muscle fibers and are sampled in muscle biopsies. Upon injury to a single myofiber, the satellite cells are activated to divide and migrate from beneath the basement membrane. These cells divide extensively, forming hundreds of myoblasts that fuse spontaneously at the site of injury to repair the host myofiber. They also fuse among themselves to form new myofibers to substitute for lost function. Furthermore, the signals to stop myoblast division and to initiate myotube formation appear to be cell confluence and low serum level. This system is harnessed in desirable embodiments.

As seen in the work of Dr. Peter Law, a factor affecting success of MTT is the age of the animal from which the cells are taken. For example, in young rats, approximately 11% of all skeletal myonuclei belong to satellite cells, declining to about 6% in the aged. In human beings past age 26 there are less satellite cells, each with shorter telomeres. Importantly, it was realized that the muscle biopsies of such human beings yield less satellite cells that also exhibit less proliferative vigor in cell culture. Accordingly, embodiments of the invention alleviate this problem by: a) selecting the allograph technique when reasonably possible and using muscle cells from other robust humans; b) selecting muscle tissue that has a higher proportion of long teleomere satellite cells; c) culturing larger amounts of biopsies (at least 0.5 gm, 1 gm, 2 gm, 3 gm, 5 gm or more than 10 gm) to start with larger numbers of cells that need to divide less often to make up a mass for injection; and d) increasing survival of transplanted cells by the use of angiogenic factor(s) as described herein.

In sum, bioengineering the regenerative heart provides novel treatments for cardiovascular diseases. Through endomyocardial injections of cultured skeletal myoblasts, the latter spontaneously transfer their nuclei into cardiomyocytes to impart myogenic regeneration. Donor myoblasts also fuse among themselves to form new myofibers, depositing contractile filaments to improve heart contractility. These myofibers contain satellite cells with regenerative vigor to combat heart muscle degeneration.

Cell Transplantation

Generally, in many embodiments, a muscle sample (or other sample) that contains muscle progenitor cells such as satellite cells is obtained from a living person. The sample tissue is disintegrated to release individual cells and the cells are grown up to a large mass. Preferably the cells are sampled and grown in a manner to avoid fibroblast overgrowth.

In a desirable allogenic embodiment, human myoblasts are obtained from a donor by sampling muscle tissue and expanded in cell culture. According to this procedure, as described by publications and a pioneering patent by Dr. Peter Law, satellite cells from a biopsy preferably are grown to a purity of at least 75%, preferably at least 85%, 90%, 95%, 97%, 99% or even greater purity, as measured by percent of non-myogenic cells. Other groups have reported preparing such cultures for clinical implantation studies, including workers at those Diacrin and at Duke University. Preferably the cells are at least 90% pure with respect to fibroblast contamination.

A number of laboratories have reported the successful purification of cells for this therapy. For example, Tremblay, U.S. No. 5,833,978, entitled “Method of in vitro preconditioning healthy donor's myoblasts before transplantation thereof in compatible patients suffering of recessive myopathies like muscular dystrophy, for improving transplantation success” claims to have achieved such purification and further allegedly useful techniques in this area. Blau et al, U.S. Pat. No. 5,538,722, entitled “Isolation, growth, differentiation and genetic engineering of human muscle cells” also apparently describes a related technique. Booth et al. apparently describes useful related techniques in U.S. Pat. No. 5,466,676, entitled “Satellite cell proliferation in adult skeletal muscle.” Also see the techniques taught by Miller in U.S. Pat. No. 6,337,184 entitled “Molecular marker for muscle stem cells.” Each of these documents is especially incorporated by reference in its entirety entireties. Most specifically, the portions of each pertaining to manipulation of muscle cells, and progenitors of muscle cells, such as for example, adding and expressing an exogenous gene in such cells, is particularly incorporated by reference specifically. The taught materials and methods are intended embodiments of the invention disclosed herein, as space and time limitations preclude importing specific details from these U.S. patents into the present specification.

In one exemplified and desirable embodiment a 2 gram muscle biopsy (0.4 to 10 grams, preferably 1 to 3 grams) from the quadriceps of a young normal human of age between 13 to 36 (and preferably between 13 and 26 years old) is taken, and satellite cells cultured. Typically, some 1,000 to 250,000 and more generally 3,000 to 30,000 satellite cells are released and cultured to form more than 500 million, preferably at least 1 billion or even more than 3, 5, 10, 25, 50, 100 or 2500 billion myoblasts. From a sample that yields 10,000 satellite cells, culturing typically causes the formation of approximately 50 billion pure myoblasts in 45 days. The myoblasts, (preferably 100 to 10,000 million, more preferably 250 to 2,000 million) are injected into the heart or other muscle or organ. To stimulate satellite cell division, it is desired to injure the muscle prior to sampling by multiple needle probings, sonication, and/or the like.

In a desirable embodiment of the invention, MTT is used to bioengineer the regenerative heart from a patient who is expected to require heart regeneration in the future or who has a damaged heart. In one embodiment between 0.5 gm and 50 gm, preferably at least 1 gm, also preferably between 2 gm and 10 gm and most preferably about 5 grams of muscle biopsies are taken from skeletal muscle (such as from both quadriceps) of a patient. The cells are cultured into approximately one billion myoblasts in 4 weeks and then injected or surgically implanted between the vascularized and the non-vascularized infracted myocardium. As described above, a number of laboratories apparently have suitable techniques for carrying out this procedure.

Tissue rejection generally is alleviated by the use of autograph transfer (cells from the same individual transplanted back) or by use of cyclosporine, in the case of allograph (cells from another individual) or xenograft (least desirable, cells from another species such as a pig). Autograft transfer often is preferred where the patient's cells are genetically normal with respect to muscle functioning, and the tissue (usually heart) is not damaged or strongly damaged. An allograft is particularly desirable for implantation into an older person such as someone over 30, 40, 50, 60, or over 70 years old. In an embodiment, the patient takes oral cyclosporine as immunosuppressant for typically 4, 5, 6, 7 or 8 weeks (preferably 4-6 weeks, or 6 weeks) to suppress rejection of the allografts. Since myoblast fusion completes within three weeks after MTT, and since myotubes and mature myofibers do not express MHC-1 surface antigens, it is not necessary to administer life-long immunosuppression as in heart transplants. Accordingly, in many embodiments allographic transfer is most desired, and can take advantage of standardized tissue samples that may serve for implantation into multiple recipients.

A common pitfall of myoblast culture is fibroblast contamination. Since myoblast doubling time is 21 hours and fibroblast doubling time is 15 hours, fibroblast growth often overtakes the myoblast culture. Fibroblasts do not deposit contractile filaments but will produce scars. From previous dose response studies in muscular dystrophies, it is estimated that the dose of about one billion (e.g. 0.2 to 20 billion, preferably 0.4 to 2.5 billion) pure myoblasts is optimal to produce the regenerative heart. Purity in this context means at least 85%, (less than 15% other cell types such as fibroblasts), preferably at least 90%, more preferably at least 95% and most preferably at least 98% myoblasts.

It was discovered experimentally that approximately one billion myoblasts (e.g. 100 million to 5 billion, preferably 200 million to 2.5 billion, more preferably 500 million to 2 billion) can be administered into a heart the size of an average adult human or pig at a suspension concentration of about 100 million myoblasts per ml of suspended cells (e.g. 20 million to 300 million, preferably 30 million to 250 million, more preferably 50 million to 200 million). Preferably the cells are injected into the wall of the heart in separate injections of about 0.1 to 1.5, more preferably 0.2 to 1 and even more preferably 0.25 to 0.6 ml injection volumes of suspended cells. Preferably between 2 to 100, more preferably between 4 to 50 and more preferably between 10 and 35 injections are made for a given heart treatment.

Desirably each injection is via a needle that protrudes less than 10 mm, more preferably less than 7.5 mm and yet more preferably less than 5 mm into an adult heart muscle wall. Depending on the site of injection the maximum distance may be altered. For preventative treatment of a healthy heart, the depth may be greater than for restorative treatment of a diseased heart for example. For children and infants, the protrusion depth is correspondingly less, as determined by the actual or estimated wall thickness. Injection preferably is via a catheter. A desirable catheter and system are described in U.S. No. 60/231,880, filed Sep. 12, 2000 and PCT/US01/28712, filed Sep. 11, 2001, the contents of which specifically are incorporated by reference in their entireties. A desirable, previously known catheter that may be used is the NOGA (TM) system from Biosense Webster, Inc.

In another embodiment skeletal myoblast—cardiomyocytes are cultured together under conditions that allow cell fusion to form heterokaryotic cardiomyocytes, which are introduced into the heart to be treated. Co-culturing may occur by mixing the two types of cells in culture. A mixing ratio of between 10% to 90% myoblasts (the remainder cardiomyocytes) is desirable. Of course, small amounts of other contaminating cells such as fibroblasts may exist, but preferably such contaminants, on a wet weight basis, comprise less than 10%, 5%, 3%, 2% or even less than 1% of the total living cell cultured material. Without wishing to be bound by any one theory for how this embodiment of the invention operates, it is believed that the cardiomyocytes (or raw cardio cellular tissue biopsy, as may be used) produce cellular factors that encourage the myoblasts to become more cardiomyocyte like. In another embodiment the cardiomyocytes fuse with the myoblasts.

In another embodiment the cultured cardiomyocytes (or a more original heart biopsy used without extensive generation and purification of cardiomyocytes) are cultured in contact with the same cell culture media but without cellular contact with myoblasts. For example, both cell types may be separated by a screen, grid, porous ceramic, membrane, immobilization on different solid phases or the like in a manner that allows cellular factors produced from the cardiomyocytes to contact the myoblasts.

Use of Skeletal Myofibers with Heart Myofibers

The beat of a heart has a myogenic origin and is initiated by pacemaker activity in the sinoatrial node. As depolarization sweeps through the atrioventricular node, the depolarization excites the Purkinje fibers of the bundle of His, which in turn signals the ventricles to contract rhythmically. Heart function would be impaired if the rhythmic action potentials do not synchronize the fiber contractions. In the regenerative heart where new skeletal myofibers are added according to embodiments presumably at different regions of the left ventricle, such heterogeny in some cases may create undesirable electric aberrant such as arrhythmia. Excitation of the heterokaryotic cardiomyocytes generally remains unchanged because there is little change in gap junctions for current flow.

The threshold of excitatory depolarization for heart and skeletal myofibers is similar, i.e., between 40 to 50 mV. Whereas the cardiomyocyte action potential is triggered with an increase in Ca²⁺ conductance into the cell, the skeletal myofiber action potential is triggered with an increase of Na⁺ conductance. Because Ca²⁺ has greater ionic size than Na⁺ and therefore lower ionic mobility, the action potential of cardiomyocytes has a longer duration (˜250 ms) than that of skeletal myofiber (˜1.5 ms). This difference in durations is advantageous for embodiments of the invention because the cardiomyocyte depolarization can continually excite the myofibers that are skeletal in origin. Since the action potentials of skeletal myofibers are of short duration, they merge into the compound action potential of the heart. The skeletal myofibers cease to fire and stop contracting once hyperpolarization of the myocardium reaches approximately −50 mV.

In an additional embodiment, a pharmaceutical compound that alters hyperpolarization is used to further assist electrical incorporation of myoblast cells into living tissue. In yet another embodiment the compound is added via local delivery via a patch or implanted reservoir near the treated heart. In yet another embodiment myogenic cells are added that have been altered morphologically or transgenically to decrease activity or numbers of sodium channels and increase the numbers of calcium channels to further improve their integration into the heart. In yet another embodiment myogenic cells grown in culture for transplant express one or more humoral substances from the heart, which alter the myogenic cells. In an embodiment the myogenic cells are grown in the presence of cardiac cells obtained from another human.

Skeletal myofibers adapt to the frequency of electric excitation to which they are subjected. Accordingly, in an embodiment, incipient myofibers (fibers that are developing but can still integrate into target heart muscle, along with myoblasts upon transfer) are subjected to entraining electrical excitation in vitro prior to transfer. The entraining pulses may be used for at least 3 hr, 6 hr, 24 hr, or more than 24 hours. Preferably the myofibers and/or myoblasts that form myofibers are incubated in the presence of cardiomyocytes to further condition them for implantation into a heart.

In the heart mileau and under the influence of heart hormones and slow contractile activity, the skeletal myofibers further develop characteristics of cardiomyocytes. In an embodiment of the invention, skeletal myofibers are combined with cardiac myofibers in a mass ratio of less than 10 to 1, preferably less than 5 to 1, 2 to 1, and even more preferably in a ratio of less than 0.1 to 1. The ratios of fiber described here desirably effects an improved heart performance. In a related embodiment, nerve cells are further added along with nerve growth factor and/or other factor as desired under conditions that facilitate nerve cell interconnection and innervation of new myofibre. In yet another embodiment, myogenic cells begin to form myotubes prior to injection. In this case, the myotubes continue to develop during and after transplantation.

Co-Use of Angiogenesis, Migratory and/or Scaffolding Cellular Integration Factors

A variety of factors may be used in combination with MTT to improve cell transplant therapy such as angiogenesis, migratory attractants and scaffold (myoblast binding/immobilization) proteins. The factors described herein may be added exogenously. For example, the factors may be targeted to heart by conjugation with a ligand that binds heart, administered by IV, or added to a cell suspension prior to implantation of the cells. For factors that are released slowly, it is preferred that the efflux occur over at least 3 days, 1 week, 2 weeks, 4 weeks or even longer. That is, at least 50% of the total amount of factor would become available and diffuse over this length of time.

The factors described herein for facilitating cell transplant therapy may be slowly released. The factors can be loosely bound by a variety of slow release technologies including for example, the salt composition complexes taught by Igari et al. (U.S. Pat. No. 6,376,461) and Johnson (U.S. Pat. No. 6,051,259 polymeric matrix of a biocompatible polymer and particles of biologically active, metal cation-stabilized hGH, wherein said particles are dispersed within the biocompatible polymer); hydrogels such as those made from poly(vinyl alcohol) (see U.S. No. Pat. 6,231,605); collagen, polyacrylamide, and the like. Most desirably the factors are included in a gel or resin material that can be implanted on the muscle surface by injection with a syringe to the desired area. In another embodiment the syringe injects the materials into the target muscle(s) itself, and slowly leaches out.

An “effective amount” of each factor is used that causes a desirable effect. The dosage of any specific integration factor depends on many factors that are well known to those skilled in the art. They include for example, the route of administration and the potency of the particular compound. The potency may be determined by routine experimentation. An exemplary dose is from about 0.001 .mu.M/kg to about 100 mg/kg body weight of the patient. Without further elaboration, it is believed that one skilled in the art can, using the preceding description, practice the present invention to its fullest extent.

Angiogenesis Factors—According to an embodiment, one or more angiogenesis factors are added to the site of implantation by one or more techniques. During the past three decades a large variety of so-called angiogenesis factors have been discovered and characterized in more detail. These are major contributors to angiogenesis. The term angiogenesis factors includes other functionally heterogeneous molecules. The best characterized angiogenesis factors are endothelial growth factors, such as “vascular endothelial growth factor” (VEGF), “fibroblast growth factor” (FGF), “platelet-derived growth factor” (PDGF), angiogenin and Interleukin-8 (IL-8). However, members of the family of matrix metalloproteinases are also included.

The list of factors involved in angiogenesis and their receptors are increasing steadily. For example, angiogenesis factors often are released from tumor cells and are studied as molecularly defined therapeutic targets. The Anton Wellstein research group in Germany, for example, has purified a novel heparin-binding polypeptide growth factor (pleiotrophin, PTN) from supernatants of breast cancer cells and cloned the respective genomic and cDNA. The respective protein is secreted from different human tumor cells, is expressed in a number of primary human tumors (breast, prostate and lung cancer and melanoma), and can function as an angiogenesis factor. The gene for this protein and others similarly discovered may be expressed transgenically in implanted myoblasts.

Further desirable proteins included within the definition of “angiogenesis factor” are those that increase the biological effects of other angiogenesis factors. For example, see Harris V K, et al. (Serum induction of the fibroblast growth factor-binding protein (FGF-BP) is mediated through ERK and p38 MAP kinase activation and C/EBP-regulated transcription) in Oncogene. 20:1730-1738, (2001); Tassi E, et al. (Enhancement of Fibroblast Growth Factor (FGF) Activity by an FGF-binding Protein) in J Biol Chem. 276:40247-40253, (2001) and Reiter R, et al. (An isoform of the coactivator aib1 that increases hormone and growth factor sensitivity is overexpressed in breast cancer) J Biol Chem. 276:39736-39741, (2001). One or more genes encoding such factors desirably are added, preferably with a constitutive promoter, to myoblasts used for transplantation, and/or the protein factor may added in the cell suspension.

Yet another desirable technique is to add one or more angiogenesis factors by separate administration prior to, during or after cell transplantation intervention of a heart. In one technique the factor is conjugated to a targeting moiety and administered to the heart, near to the heart, or systemically for a period of time (a day, two days, a week, two weeks etc) before transplantation to allow build up of vessels in the targeted tissue. The angiogenesis factor(s) may be prepared and added as factors in the myoblast cellular suspension itself. For example, a suitable concentration may be determined from the literature and/or determined by routine experimentation using, for example, 0.1 ng/ml, 1 ng/ml, 10 ng/ml, 100 ng/ml of protein per ml of cell suspension, injection solution and the like. When added to the cell suspension, the factor may be added as a pure or partly purified material. The factor may be added indirectly by incubating non myoblast cells that produce the factor with myoblast cells, or having them share the same incubation fluid (separated by screens, for example) so that the factor producing cells are not harvested and injected with the myoblasts.

Factor producing cells may be used to seed a culture of myoblasts to be injected and may be injected along with the myoblasts. This latter technique is particularly suitable where the factor producing cells do not form scar tissue, and (preferably) do not survive for long (more than one day, one week or several weeks for example) after transfer. The angiogenesis factor(s) may be added to the heart by separate injection of a solution, gel, colloid or other form of the factor(s), before, during and/or after administration of myoblasts. Polymeric substances may be used to entrap such proteins and other factors. In one embodiment, a factor is attached covalently to a polymer or other material that is placed onto muscle tissue (such as heart) or that binds to the tissue after administration intraveneously, or other way. A skilled artisan can adjust relevant factors to cause gradual release or availability of the angiogenesis factors to the muscle.

Most desirably, one or more genes that encode angiogenesis factor(s) are incorporated into a cell for transient or permanent genetic expression. When it is desired to have limited expression, transient expression may be obtained by placing one (or preferably many copies of the) gene into a myoblast but outside the nuclear genome. Such transient expression may occur through use of a viral vector or other procedure that adds nucleic acid to the cell.

Without wishing to be bound by any one theory for how embodiments of the invention operate, cell transplantation as described herein, provides myoblasts that survive, develop and function as “aliens” in the heart. This integration is strongly influenced by a rich set of complex biological interactions involving the supply of nutrients to the new cells, binding/immobilization of the new cells and, in some cases, migration of transplanted cells. The myocardial aliens turn into are newly formed skeletal myofibers that contribute to cardiac output through production of contractile filaments after settling in. The nuclei are donor in origin and as skeletal myofibers, will have satellite cells and regenerative capability. The cardiomyocyte aliens are donor myoblast nuclei carrying chromosomes that preferably have long telomeric DNA subunits that are essential for mitosis. Upon injury of this heterokaryotic cardiomyocyte, the myoblast regenerative genome activates, producing foreign contractile filaments such as myosin. Each step in the transplant and subsequent integration and use of the new cells may be positively influenced by one or more biological factors. In recognition of this fact, several desirable alternations in biological factors are presented herein to improve chances of transplanted cell survival and use by the recipient tissue.

Accordingly, one or more substances may be added to facilitate the integration and use of transplanted cells. Desirably, molecular agents that help the transplanted cells coordinate excitatory depolarization are used, and may be for example, transgenically expressed in a transplanted cell, or added as a slow release agent at the site of transplantation. Such agents may be present in a slow release matrix such as loosely bound in a gel, colloid, or other material at or near the implantation site, or may be covalently bound to a material near or at the site, and slowly released by action of an enzyme, such as an enzyme normally thought to be active at that region. Other factors may be added, as reviewed next.

Migration, scaffolding factors Most desirably, implanted target tissue is labeled by adding migratory attractants, in a preferably leachable form, which create a concentration gradient suitable for the implanted or injected myoblasts to follow. The attractants may be added to implanted patches, glues or the like, which may comprise a gel, hydrogel, complex surface, colloidal space, etc. that has leachable myoblast attractants, which slowly dissipate, creating a signal for myoblasts to follow. Myoblasts may be injected nearby or systematically, find such surfaces and can fuse with muscle tissue there, preferably induced by one or more differentiation factors, and/or bind to scaffold proteins there such as fibronectin, and settle down.

The attractants can be added to an implant surface by planting a substance, such as a patch, glue, gel or other material that stays at the muscle surface, but which slowly releases the attractants. Migratory myoblasts that are attracted to the muscle surface can fuse with and add bulk to the muscle. This is particularly useful to direct cells to areas where it is desired to build up bulk.

Migratory factors contemplated include, for example, crude extracts of injured muscle tissue, such as a water soluble low molecular weight extract from minced muscle that has been allowed to sit in culture media for 5 hours after mincing. This kind of extract can be prepared with a 30,000 molecular weight or 100,000 molecular weight cutoff filter. Within such kind of extract a number of proteins and other factors can be purified that can act as attractants. U.S. Pat. No. 6,284,242 issued to Kurachi on Sep. 4, 2001 describes the use of basic fibroblast growth factor and fibronectin in this context. U.S. application No. 20010055590 (Dec. 27, 2001) to this same group further describes desirable factors such as cytokines that may be used in this embodiment. In particular, PDGF, HGF, fibronectin, MMP-1 and MMP-2 may be manipulated and used, as for example described for migration of myogenic precursor cells during development See (Daston et al “Pax-3 is necessary for migration, not differentiation, of limb muscle precursors in the mouse” Development 122:1017-1027, 1996; Bladt et al. “Essential role for the c-met receptor in the migration of myogenic precursor cells into the limb bud” Nature 376:768-771, 1995; Venkatsubramanian and Solursh “Chemotactic behavior of myoblasts” Devel Biol 104:406-407, 1984; Krenn et al. “Hyaluronic acid influences the migration of myoblasts within the avian embryo wing bud” Am J. Anat 192:400-406, 1991; Brand-Saberi et al. “Differences in fibronectin-dependence of migrating cell populations” J Embyol 187:17-26, 1993; and Chin and Werb “Matrix metalloproteinases regulate morphogenesis, migration and remodeling of epithelium, tongue skeletal muscle and cartilage in the mandibular arch” Development 124:1519-1530, 1997.

In another desirable embodiment, extracellular matrix protein that binds, preferably specifically to myoblast cells and/or to mature muscle tube surface is added to the site of cellular implantation before (or less desirably during or after) implantation. A variety of myoblast scaffold proteins are contemplated that can be affixed to the surface by a variety of techniques. These proteins include laminin, laminin-1, fibronectin, a collagen, type I collagen, type II collage, type IV collagen, thrombospondin-I, lecithin-oxytetracycline-collagen matrix, a galactin, galectin-1, vitronectin, and von Willebrand protein.

A System Approach: Key to Large Scale Commercial Success

The optimal use of myogenic cell transplantation therapy for a specific disease condition such as heart disease requires a review of the multiple facets involved. Cells that are implanted should be as pure as possible. The cells may be obtained from the patient, a relative or other human, or even a non-human animal, as desired according to a particular situation such as the quality of the patient's genome, the age of the patient, and time available before a transplant is to be carried out. Cells to be implanted may be conditioned or even fused with cardiomyocytes. Other factors such as migratory agents, attractants, scaffolding proteins, angiogenesis factors and so on most preferably are combined to improve long term prognosis. A variety of agents and methods for their delivery have been reviewed.

The basic technique of myoblast transfer therapy is accepted and a variety of laboratories claim to have useful procedures for obtaining suitable cells. In fact, the first human myoblast transfer into the porcine heart already has revealed the safety of to administering one billion myoblasts at 100 million/mL through a Myostar catheter (Biosense Webster Inc.) using 20 injections at different locations inside the left ventricle. See P K Law, et al., “World's First Human Myoblast Transfer into the Heart”, Frontiers in Physiology, (2000), p. A85. For the particular conditions evaluated in that study, 0.3 to 0.5 mL injection volumes were deemed optimal. A further systems' approach involves the need to automate and inject cells more reproducibly. In particular, U.S. patent application No. 60/231,880 filed Sep. 12, 2000, the contents of which specifically are incorporated by reference in its entirety and entitled “Myogenic Cell Transfer Catheter and Method” describes a most useful injection system that is designed particularly for use in combination with embodiments detailed herein.

Because of the sheer magnitude of heart disease in industrialized countries, a huge demand is expected to exist for normal myoblasts of sufficient purity as mentioned herein. Accordingly, further embodiments of the invention are intended to be practiced in combination with an automated cell processor. Most desirably, U.S. Pat. No. 6,261,832 “Automated Cell Processor” issued to Peter Law describes an apparatus that minimizes the labor intensiveness and high cost of cell culturing, harvesting and packaging, and the fallibility of human imprecision. This device can manufacture large quantities of viable, sterile, genetically well-defined and functionally demonstrated biologics, examples of which are myoblasts and myoblast-derived heterokaryons.

The automated cell processor combines computer technology, mechanical engineering, and cytogenetics. In variations of this apparatus, the machine accepts biopsies of various human tissues. The machine includes a computer that can be programmed to process tissue(s), with precision controls in time, space, proportions of culture ingredients and apparatus maneuvers. Cell conditions may be monitored at any time during the process and flexibility is built-in to allow changes. Different protocols may be programmed into the software for culture¹⁰, controlled cell fusion¹¹, harvest and package. The outputs supply injectable cells ready for cell therapy or shipment. The cell processor can be self-contained in a sterile enclosure large enough to house the hardware in which cells are cultured and manipulated. The automated cell processor can replace bulky inefficient culture equipment, elaborate manpower, and mistakes from the manpower now used for cell culture. The machine can de-centralize cell production, allowing the latter to be conducted in hospitals where transport of patients' muscle biopsies and the autologous myoblasts is cut to a minimum.

Modifications to this instrument are contemplated as embodiments of the invention. For example, myoblasts and cardiomyocytes may be inputted as separate reagents and incubated together in the system. Another important embodiment is the automated transgenic incorporation of desired gene(s) such as an angiogenic factors into cultured cells within this machine. For example, one reagent of the automated processor can be a vector, such as a virus vector that contains a gene for a human VEGF-165 gene. Preferably the vector contains a linked marker gene and the automated instrument automatically selects for successful transformation by incubation of an inhibitor compound that inhibits growth of cells that lack an expressible protective marker. Yet another representative embodiment is an automated quality control step in the instrument that automatically scores cell cultures for the presence of fibroblast cells, by detecting fibroblasts directly or indirectly. An example of the latter is to add a labeled antibody that specifically binds to the surface of fibroblasts. A representative automated purity assay may involve adding fluorescently labeled anti-fibroblast antibody, rinsing away unbound conjugate, and measuring the total amount of fluorescence remaining, as an index of how many fibroblast cells are present.

Yet other powerful systems technology intended as embodiments of the invention include linking diagnostic tests with use of cell transplantation therapy. A representative technology is this context is represented by the CardioChip, which allows early diagnosis of cardiovascular diseases using 10,368 expressed sequence tags (ESTs). J D Barnes, D Stamatiou, C C Liew, “Construction of a Human Cardiovascular cDNA Microarray: Portrait of the Failing Heart”, Biochem Biophy Res Comm, 280 (2001), pp. 964-969. A particularly desirable method, for example, is to obtain a nucleic acid from a subject, scan the sample for the presence of known genetic alleles and/or genetic diseases using the CardioChip (or other screening test method) that particularly relate to genetic defects in muscle function and/or lipid metabolism leading to heart damage. Subjects identified as having a genetic anomaly via the screen can have muscle biopsy taken before any symptom occurs. Myoblasts with an acceptable genetic profile can be processed and deposited in a cell bank for future HCT or be injected into the subject to prevent a problem such as a sudden heart attack or blood disorder. Other basic uses of embodiments will became apparent to a skilled artisan reader and are contemplated.

### EXAMPLE 1

Human myoblasts were manufactured as described by U.S. Pat. No. 5,130,141. J D Barnes, D Stamatiou, C C Liew, “Construction of a Human Cardiovascular cDNA Microarray: Portrait of the Failing Heart”, Biochem Biophy Res Comm, 280 (2001), pp. 964-969. Myoblasts were 90% pure as determined by desmin staining. Repeated transductions (3×) of the myoblasts with retroviruses carrying Lac-Z yielded highly efficient 70-75% Lac-Z positive cell population. A dye exclusion test using trypan blue revealed over 95% cell viability at the time of injection in NUH.

The following procedure was then conducted with a license of the Singapore Patent No. 34490 (WO 96/18303). P K Law, “Myoblast therapy for mammalian diseases”, Singapore Patent No. 34490 (WO 96/18303), issued Aug. 22, 2000. A porcine heart simulation of chronic ischemia was created by clamping an ameroid ring around the left circumflex coronary artery in Yorkshire swine, four weeks prior to cell transplantation. For cell transplantation, the animals were anesthetized and ventilated, and their hearts exposed by left thoracotomy. Fifteen injections (0.25 ml each) containing 300 million cells total were injected into the left ventricle endocardially under direct vision. For control animals, only culture medium without cells was injected. The animals were euthanized, and their heart explanted and processed for histological examination. Tissues were cryosectioned. Subsequent staining for Lac-Z expression, Hematoxylin-Eosin staining, Mason trichome staining and immunostaining for skeletal muscle myosin heavy chain were carried out by standard methods.

Histological examination of explanted porcine myocardium after 10 weeks revealed not only myofibers of human origin, but also porcine cardiomyocytes having human myonuclei with Lac-Z gene expression. More than 80% of the Lac-Z positive porcine cardiomyocytes immunostained positive for human myosin heavy chain. Control muscle stained sections did not show any Lac-Z expression nor human myosin immunostain.

The data indicated that human myoblasts survived and integrated into the porcine ischemic myocardium, allowing concomitant cell therapy and genome therapy. New fiber formed in the heart and improved heart contractility.

### EXAMPLE 2

This example demonstrates the use of MTT to repair a heart from a heart attack patient. In this example, MTT is carried out using 5 gms of muscle from a patient as described in the standard operating procedures as described in U.S. No. 60/231,880. The treated heart is found to be stronger as a result of the treatment.

### EXAMPLE 3

This example demonstrates the use of MTT to repair a heart from a heart attack patient. In this example, MTT is carried out using 5 gms of muscle from a patient as described in the standard operating procedures except that cells obtained from a human myocardium are cultured with the myoblasts during expansion of the skeletal cell biopsy into larger numbers of cells. Myocardium cells are obtained by biopsy from another human and cultured at a ratio (nuclei or cell number ratio) with the cultured myoblasts of 1 to 100. The co-culturing continues for 4 days, after which the myoblasts are found to be more conditioned for cardiac transplant. Prior to transplant, the myoblasts are separated from the co-cultured cells, and then processed and injected into a heart as described in the standard operating procedures. The co-cultured cells are found to be more efficacious in reversing the effects of heart attack.

### EXAMPLE 4

This example demonstrates the use of MTT to prevent heart damage in a patient with a poor heart prognosis. A patient with known heart weakness is treated as described in Example 2. One year after receiving the MTT therapy the treated heart displays a stronger physiology.

### EXAMPLE 5

This example demonstrates the use of pharmaceutical agents to improve the efficacy of MTT therapy of heart. A weak, damaged heart is treated as described in Example 2, except that VEGF (vascular epidermal growth factor transduction) is added to the MTT transplant medium before injection into the treated heart. The added chemical improves the ability of the introduced cells to integrate and/or contract in unison with the pre-existing cardiocyte cells. This example is repeated with 100 ug/ml chondroitin sulfate and similar improvements are obtained.

### EXAMPLE 6

This example demonstrates the use of transgenic expression of angiogenesis factor(s) in transplanted cells for improved heart augmentation therapy via comcomitant angiogenesis/myogenesis.

A porcine heart model of chronic ischemia (control=3, myoblast-implanted=6) was produced by clamping an ameroid ring around the left circumflex artery. Four weeks later, the heart was exposed by left thoracotomy and myoblast cells implanted. The myoblast cells were cultured myoblasts from satellite cells derived from human rectus femoris biopsies. The human myoblasts were transduced with retroviral and adenoviral vectors that carry Lac-Z and human VEGF-165 genes, respectively. The cells were characterized for VEGF-165 transduction and expression efficiency by immunostaining, enzyme-linked immunosorbent assay (ELISA), immunoblotting and RT-PCR. The transduction efficiency for Lac-Z and VEGF-165 was 75% to 80% and >95%, respectively. The transduced myoblasts continued to secrete VEGF-165 for longer than 18 days, which was significantly higher (37+−3 ng/ml) than non-transduced myoblasts (200+−30 pg/ml). A dye exclusion test revealed >95% cell viability at the time of injection.

Twenty injections (0.25 ml each) containing 300 million (total) myoblasts or 5 ml total volume of basal Dulbecco's Modified Eagle's Medium (DMEM) as control were injected into the left ventricle intramyocardially. Left ventricular function was assessed using MIBI-Tc-99m single photon emission computed tomography (SPECT) scanning one week before injection to confirm myocardial infarction and at six weeks after injection.

Animals were maintained on cyclosporin at 5 mg/kg body weight from five days before until six weeks after cell transplantation. The animals were euthanised at six weeks to five months post-operatively, and the hearts were processed for histological, immunocytochemical and ultra-structural studies.

Histological examination showed extensive survival of the grafted myoblasts expressing Lac-Z gene in and around the infart. More than 80% of the Lac-Z positive cardiomyocytes immunostained positively for human myosin heavy chain. The control heart without myoblast injection did not show Lac-Z positive myonuclei nor human myosin. A triple stain of myoblast-injected myocardia revealed multinucleated heterokaryons containing human and porcine nuclei with expression of human myosin. Electron microscopy demonstrated human myotubes and skeletal myofibres with satellite cells in the porcine myocardium. Laser nuclear capture, together with single nucleus reverse transcription polymerase chain reaction (RT-PCR), was performed to delineate host and donor nuclei. In situ hybridization using fluorescent DNA probes specific to human Y-chromosomes and chromosomes 1 and 10 for pigs were used.

The vascular density (mean+−scanning electron microscopy (SEM) counted in an average of 12 low power fields (×200) in control animal hearts was 4.18+−0.42, compared with the VEGF-165 myoblast transplanted group (28.31+−1.84). The SPECT scans showed improved perfusion in the infarcted region. Discontinuation of cyclosporine after six weeks prompted no xenograph rejection for up to 20 weeks.

Each publication and patent application cited herein is incorporated in its entirety by reference.

I claim:

1. A method for producing cardiomyocytes capable of proliferation, comprising:
(a) providing cardiomyocyte cells;
(b) providing myoblast cells; and
(c) mixing the cells of step (a) with the cells of step (b) under in vitro or in vivo conditions that allow cell fusion of cardiomyocyte cells with myoblast cells to form heterokaryotic cardiomyocytes.

2. A method as described in claim 1, further comprising a selection step wherein cells are selected based on their abilities to proliferate.

3. Heterokaryotic cardiomyocytes produced by the process described in claim 1.

4. A method as described in claim 1, wherein the selection step comprises the detection of mitosis.

5. A method as described in claim 1, wherein step (c) comprises the addition of chondroitin sulfate.

6. A method as described in claim 5, wherein the chondroitin sulfate is added to a final concentration of between 5 micromolar to 5 millimolar.

7. Heterokaryotic cardiomyocytes produced by the process described in claim 1.

8. A method of producing human heterokaryons exhibiting the characteristics of both myoblasts and cardiomyocytes, comprising:
(a) culturing human myoblast cells from one or more human biopsies;
(b) providing cardiomyocyte cells; and
(c) incubating the cells from step (a) with the cells of step (b) under conditions that allow fusion of human myoblasts with cardiomyocytes.

9. A method as described in claim 8, wherein step (a) is carried out by culturing the human myoblasts through at least one mitosis.

10. A method as described in claim 9, further comprising a selection step wherein one or more clones are selected based on the abilities of the heterokaryons to proliferate.

11. A method as described in claim 9, wherein the selection step comprises detection of mitosis.

12. Heterokaryotic cardiomyocytes produced by the process described in claim 8.

13. A method as described in claim 8, wherein step (c) comprises the addition of chondroitin sulfate.

14. A method as described in claim 13, wherein the chondroitin sulfate is added to a final concentration of between 5 micromolar to 5 millimolar.

15. Heterokaryotic cardiomyocytes produced by the process described in claim 12.

16. A method of replenishing degenerated and degenerating cardiomyocytes of a patient with heart disease, comprising:
(a) providing heterokaryotic cardiomyocytes capable of developing desmosomes and gap junctions; and
(b) administering the heterokaryotic cardiomyocytes of step (a) through a catheter pathway.

17. A method as described in claim 16, wherein the cardiomyocytes of step (a) are prepared by the additional step of controlled cell fusion in vitro between myocytes and cardiomyocytes.

18. A method as described in claim 16, wherein the controlled cell fusion step comprises the addition of chondroitin sulfate.

19. A method as described in claim 16, wherein the chondroitin sulfate is added to a final concentration of between 5 micromolar to 5 millimolar.

20. A composition of cells useful for repair of damaged heart muscle, comprising heterokaryons that exhibit characteristics of both normal myoblasts and normal cardiomyocytes, including the ability to undergo mitosis in vitro and to develop desmosomes, gap junctions, and to contract in synchrony after transplantation into damaged heart muscle.

21. A composition as described in claim 20, further comprising between 5 micromolar to 5 millimolar chondroitin sulfate.

22. A composition of cells useful for repair of damaged heart muscle, comprising heterokaryons that exhibit characteristics of both normal myoblasts and normal cardiomyocytes, including the ability to undergo mitosis in vitro.

23. A composition as described in claim 20, wherein the heterokayons transgenically express a cellular integration factor selected from the group consisting of an angiogenesis factor, TGF-beta, vascular endothelial growth factor, fibroblast growth factor, platelet derived growth factor, angiogenin, pleiotrophin, and interleukin-8.

24. A composition as described in claim 20, further comprising a cellular integration factor selected from the group consisting of a migration factor, a scaffolding protein, PDGF, HGF, fibronectin, MMP-1, MMP-2, laminin, laminin-1, fibronectin, type I collagen, type II collagen, type IV collagen, thrombospondin-I, lecithin-oxytetracycline-collagen matrix, a galactin, galectin-1, vitronectin, and von Willebrand protein.

25. A composition of cells useful for repair of damaged heart muscle, comprising myoblasts that have been transgenically transformed to express a cellular integration factor selected from the group consisting of an angiogenesis factor, vascular endothelial growth factor, fibroblast growth factor, TGF-beta, platelet derived growth factor, angiogenin, pleiotrophin, and interleukin-8.

26. A composition as described in claim 25, further comprising a cellular integration factor selected from the group consisting of a migration factor, a scaffolding protein, PDGF, HGF, fibronectin, MMP-1, MMP-2, laminin, laminin-1, fibronectin, type I collagen, type II collagen, type IV collagen, thrombospondin-I, lecithin-oxytetracycline-collagen matrix, a galactin, galectin-1, vitronectin, and von Willebrand protein.

27. A composition of cells useful for repair of damaged heart muscle, comprising myoblasts and an effective amount of a cellular integration factor selected from the group consisting of an angiogenesis factor, vascular endothelial growth factor, fibroblast growth factor, platelet derived growth factor, angiogenin, TGF-beta, pleiotrophin, and interleukin-8a migration factor, a scaffolding protein, PDGF, HGF, fibronectin, MMP-1, MMP-2, laminin, laminin-1, fibronectin, type I collagen, type II collagen, type IV collagen, thrombospondin-I, lecithin-oxytetracycline-collagen matrix, a galactin, galectin-1, vitronectin, and von Willebrand protein.
